(19) United States
(12) Patent Application Publication
(10) Pub. No.: US 2002/0028212 A1
(43) Pub. Date: Mar. 7, 2002

(54) RECOMBINANT VIRUSES CODING FOR A GLUTAMATE DECARBOXYLASE (GAD) ACTIVITY

(76) Inventors: MARIE-CLAUDE GEOFFROY, VERSAILLES (FR); PHILIPPE HORELLOU, PARIS (FR); JEAN-FRANCOIS JULIEN, ANTONY (FR); JACQUES MALLET, PARIS (FR); MICHEL PERRICAUDET, ECROSNES (FR); JEAN-JACQUES ROBERT, SCEAUX (FR); EMMANUELLE VIGNE, IVRY SUR SEINE (FR); ALEXIS BEMELMANS, PARIS (FR)

Correspondence Address: FINNEGAN HENDERSON FARABOW GARRETT & DUNNER LLP, 1300 I STREET N W, SUITE 700, WASHINGTON, DC, 20005-3315, US

(21) Appl. No.: 08/704,583

(22) PCT Filed: Mar. 21, 1995
(86) PCT No.: PCT/FR95/00342

(30) Foreign Application Priority Data
Mar. 23, 1994 (FR) 94/03411
Nov. 9, 1994 (FR) 94/13487

(51) Int. Cl. A61K 39/12

(52) U.S. Cl. 424/204.1; 424/205.1

(57) ABSTRACT

Recombinant viruses comprising a heterologous DNA sequence coding for a protein having glutamate decarboxylase (GAD) activity, preparation thereof, and therapeutic use thereof, in particular for treating and/or preventing degenerative neurological diseases.

The present invention relates to recombinant vectors of viral origin, to their preparation and to their use, especially for the treatment and/or prevention of neurodegenerative diseases. More particularly, it relates to recombinant viruses containing a DNA sequence encoding a protein having glutamate decarboxylase activity (GAD). The invention also relates to the preparation of these vectors, to pharmaceutical compositions containing them and to their therapeutic use, especially in gene therapy.

The gabaergic pathways represent the main group inhibiting the nervous system in vertebrates. Impairment of the activity of the GABAergic neurons manifests itself immediately in the whole body by dyskinesias or convulsions. Furthermore, the role of cerebral gamma-amino-butyric acid (GABA) is not limited to neurotransmission. A neurotrophic action during development has indeed been attributed to it, in particular in the neuroretina. Moreover, GABA is also present in the β cells of the islets of Langerhans, where it seems to play a role in the regulation of the production of insulin.

The possibility of restoring or installing de novo a GABA synthesis in a precise region of the body therefore has a major therapeutic advantage both for conditions directly linked to the degeneration of GABAergic neurons and for those which respond to GABA agonists. The present invention offers a particularly advantageous solution to this problem. The present invention indeed describes the development of vectors of viral origin which can be used in gene therapy, capable of expressing in vivo, in a localized and efficient manner, a glutamate decarboxylase activity. The present invention thus describes a new approach for treating conditions linked to a GABA deficiency, consisting in inducing the in vivo synthesis of GABA by targeted release of a biologically active enzyme.

Glutamate decarboxylase activity (GAD) is an enzyme which catalyses, in a relatively specific manner, the conversion of glutamate to gamma-amino-butyric acid (GABA) with the aid of a cofactor, pyridoxal phosphate (vitamin B₆). In the native state, this enzyme exists in the form of a dimer of 120 kilodaltons. After reduction of the disulphide bridges, examination by specific electroimmunoanalysis (Western blotting) reveals two bands at 65 and 67 kilodalton. It was recently demonstrated that these two monomers correspond to two different proteins encoded by distinct genes (Erlander et al., Neuron 7 (1991) 91). The two molecules, hereinafter called GAD 67 and GAD 65, differ from an enzymatic point of view, the short form having a lower affinity for pyridoxal phosphate, and by their subcellular localization, the short form being better represented in the neuronal extensions whereas the long form accumulates in the perikaryon. The long form, by virtue of its lower dependence on the concentration of pyridoxal phosphate, is therefore the one most capable of being expressed in various types of cells. GAD 65 on the other hand has the feature of becoming rapidly inactivated in the absence of pyridoxal phosphate by becoming converted to an apoenzyme, lacking catalytic activity. The reverse conversion, from apoenzyme to active holoenzyme, is relatively slow.

In addition, the expression of GAD 67 and 65 is not restricted to the nerve tissues. Both GADs also coexist in the β cells of the islets of Langerhans of the pancreas. Anti-GAD autoantibodies have thus been detected in patients suffering from a very rare neurological disease, the Stiff-man syndrome, characterized by muscular hypertonia and accompanied by degeneration of certain GABAergic neurons. A significant proportion of these patients (20%) develop, in addition, a type I diabetes. Likewise, GAD 67 is also relatively abundant in the mantle of the flagellum of spermatozoa where there is strong evidence that it plays a major role in oxidative catabolism.

The present invention consists in the discovery that it is possible to produce in vivo an enzymatic activity capable, in turn, of promoting or inducing the synthesis of a neurotransmitter, GABA. The present invention also consists in the development of vectors permitting the efficient, localized and prolonged release of an enzyme which is active in vivo on the synthesis of GABA. The present invention thus offers a new approach to gene therapy which is particularly advantageous for the treatment and/or prevention of neurodegenerative pathologies.

The applicant addressed more particularly the use of vectors of viral origin for the transfer in vivo, especially in the nervous system, of a glutamate decarboxylase activity. In a particularly advantageous manner, the applicant has now shown that it is possible to construct recombinant viruses containing a DNA sequence encoding GAD, to administer these recombinant viruses in vivo, and that this administration permits a stable, localized and efficient expression of a GAD which is biologically active in vivo, in particular in the nervous system, and without cytopathologic effect. The present invention results more particularly from the demonstration of the particularly advantageous properties of certain viruses for the expression of a GAD activity, and from the construction of viral vectors (defective viruses, deleted of certain viral regions, containing certain promoters and the like) permitting a particularly efficient expression of GAD activity, and in the appropriate tissues. The present invention thus provides viral vectors which can be used directly in gene therapy, particularly suitable and efficient for directing the expression of GAD in vivo.

A first subject of the invention therefore consists in a defective recombinant virus comprising a DNA sequence encoding a protein having a glutamate decarboxylase activity.

The subject of the invention is also the use of such a defective recombinant virus for the preparation of a pharmaceutical composition intended for the treatment and/or prevention of neurodegenerative diseases.

For the purposes of the invention, the term protein having a glutamate decarboxylase activity (GAD) designates any protein capable of inducing or promoting the production of GABA from glutamate. More particularly, the protein having a glutamate decarboxylase activity is chosen from all or part of the GAD65 and GAD67 proteins, or derivatives thereof.

The glutamate decarboxylate activity produced within the framework of the present invention may be human or animal GAD. The DNA sequence encoding GAD65 and 67 has been cloned and sequenced from various species, and in particular from man (Bu et al., PNAS 89 (1992) 2115) and from rats (Julien et al., Neurosci. Letters 73 (1987) 173). In order to allow their incorporation into a viral vector according to the invention, these sequences are advantageously modified, for example by site-directed mutagenesis, in particular for the insertion of appropriate restriction sites. The sequences described in the prior art are indeed not constructed for a use according to the invention, and preliminary adjustments may be necessary in order to obtain high expression levels. Within the framework of the present invention, it is preferable to use a DNA sequence encoding a human GAD. Moreover, as indicated above, it also possible to use a construct encoding a GAD 65 or 67 derivative, in particular a derivative of human GADs. Such a derivative comprises for example any sequence obtained by mutation, deletion and/or addition compared with the native sequence, and encoding a product conserving the capacity to induce or promote the production of GABA from glutamate. These modifications can be made by techniques known to persons skilled in the art (see general molecular biology techniques below). The biological activity of the derivatives thus obtained can then be easily determined, as indicated especially in Example 2. The derivatives according to the invention can also be obtained by hybridization from nucleic acid libraries, using as probe the native sequence or a fragment thereof.

These derivatives are especially molecules having a greater affinity for their binding sites, molecules having a greater resistance to proteases, molecules having a greater therapeutic efficiency or fewer side effects, or possibly new biological properties. The derivatives also include modified DNA sequences permitting an enhanced expression in vivo.

Among the preferred derivatives, there may be mentioned more particularly natural variants, molecules in which certain N- or O-glycosylation sites have been modified or suppressed, molecules in which one or more residues have been substituted, or molecules in which all the cystein residues have been substituted (muteins). There may also be mentioned the derivatives obtained by deletion of regions not or barely involved in the interaction with the binding sites considered or expressing an undesirable activity, and the derivatives containing, compared to the native sequence, additional residues, such as for example an N-terminal methionine and/or a secretion signal and/or a joining peptide.

The DNA sequence encoding the GAD used within the framework of the present invention may be a cDNA, a genomic DNA or a hybrid construct consisting for example of a cDNA in which one or more introns could be inserted. This may also be synthetic or semisynthetic sequences. In a particularly advantageous manner, a cDNA or a gDNA is used. In particular, the use of a genomic DNA allows an enhanced expression in human cells.

In a first embodiment, the present invention relates to a defective recombinant virus comprising a cDNA sequence encoding a protein having a GAD activity. In another preferred embodiment of the invention, the virus comprises a gDNA sequence encoding a protein having a GAD activity.

The vectors of the invention can be prepared from various types of viruses. Preferably, viruses derived from adenoviruses, adeno-associated viruses (AAV), herpesviruses (HSV) or retroviruses are used.

The viruses according to the invention are defective, that is to say that they are incapable of autonomously replicating in the target cell. Generally, the genome of the defective viruses used within the framework of the present invention therefore lacks at least the sequences necessary for the replication of the said virus in the infected cell. These regions can be either removed (completely or partly), or rendered non-functional, or substituted by other sequences and especially by the DNA sequence encoding GAD. Preferably, the defective virus nevertheless conserves the sequences in its genome which are necessary for the encapsulation of the viral particles.

As regards more particularly adenoviruses, various serotypes, whose structure and properties vary somewhat, have been characterized. Among these serotypes, the use of the type 2 or 5 human adenoviruses (Ad 2 or Ad 5) or of the adenoviruses of animal origin (see application FR 93 05954) is preferred within the framework of the present invention. Among the adenoviruses of animal origin which can be used within the framework of the present invention, there may be mentioned adenoviruses of canine, bovine, murine (example: MAV1, Beard et al., Virology 75 (1990) 81), ovine, porcine, avian or alternatively simian (example: SAV) origin. Preferably, the adenovirus of animal origin is a canine adenovirus, or more preferably a CAV2 adenovirus [Manhattan strain or A26/61 (ATCC VR-800) for example]. Preferably, adenoviruses of human or canine or mixed origin are used within the framework of the invention.

Preferably, the defective adenoviruses of the invention comprise the ITRs, a sequence allowing the encapsulation and the sequence encoding the protein having a GAD activity. Still more preferably, in the genome of the adenoviruses of the invention, the E1 gene and at least one of the E2, E4 and L1-L5 genes are non-functional. The viral gene considered can be rendered non-functional by any technique known to persons skilled in the art, and especially by total suppression, by substitution or partial deletion, or by addition of one or more bases in the gene(s) considered. Such modifications can be obtained in vitro (on the isolated DNA) or in situ, for example by means of genetic engineering techniques, or alternatively by treating with mutagenic agents.

The defective recombinant adenoviruses according to the invention can be prepared by any technique known to persons skilled in the art (Levrero et al., Gene 101 (1991) 195, EP 185 573; Graham, EMBO J. 3 (1984) 2917). In particular, they can be prepared by homologous recombination between an adenovirus and a plasmid carrying, inter alia, the DNA sequence encoding GAD. The homologous recombination occurs after co-transfection of the said adenoviruses and plasmid into an appropriate cell line. The cell line used should preferably (i) be transformable by the said elements, and (ii) contain the sequences capable of complementing the defective adenovirus genome part, preferably in integrated form in order to avoid risks of recombination. As an example of a cell line, there may be mentioned the human embryonic kidney line 293 (Graham et al., J. Gen. Virol. 36 (1977) 59) which contains especially, integrated in its genome, the left hand part of the genome of an Ad5 adenovirus (12%). Strategies for constructing vectors derived from adenoviruses have also been described in Applications Nos. FR 93 05954 and FR 93 08596.

Next, the adenoviruses which have multiplied are recovered and purified according to conventional molecular biology techniques as illustrated in the examples.

As regards the adeno-associated viruses (AAV), they are relatively small DNA viruses which become integrated into the genome of the cells which they infect, in a stable and site-specific manner. They are capable of infecting a broad spectrum of cells, without inducing any effect on cell growth, morphology or differentiation. Moreover, they do not seem to be involved in pathologies in man. The genome of the AAVs has been cloned, sequenced and characterized. It comprises about 4700 bases and contains, at each end, an inverted repeat region (ITR) of about 145 bases which serves as replication origin for the virus. The remainder of the genome is divided into 2 essential regions carrying the encapsulation functions: the left hand part of the genome, which contains the rep gene involved in the viral replication and the expression of the viral genes; the right hand part of the genome, which contains the cap gene encoding the virus capsid proteins.

As regards the adeno-associated viruses (AAV), they are relatively small DNA viruses which become integrated into the genome of the cells which they infect, in a stable and site-specific manner. They are capable of infecting a broad spectrum of cells, without inducing any effect on cell growth, morphology or differentiation. Moreover, they do not seem to be involved in pathologies in man. The genome of the AAVs has been cloned, sequenced and characterized. It comprises about 4700 bases and contains, at each end, an inverted repeat region (ITR) of about 145 bases which serves as replication origin for the virus. The remainder of the genome is divided into 2 essential regions carrying the encapsulation functions: the left hand part of the genome, which contains the rep gene involved in the viral replication and the expression of the viral genes; the right hand part of the genome, which contains the cap gene encoding the virus capsid proteins.

The use of vectors derived from AAVs for the transfer of genes in vitro and in vivo has been described in the literature (see especially WO 91/18088; WO 93/09239; U.S. Pat. No. 4,797,368, U.S. Pat. No. 5,139,941, EP 488 528). These applications describe various constructs derived from AAVs, from which the rep and/or cap genes are deleted and replaced by a gene of interest, and their use for the transfer in vitro (on cells in culture) or in vivo (directly in an organism) of the said gene of interest. The defective recombinant AAVs according to the invention can be prepared by co-transfection, into a cell line infected by a human helper virus (for example an adenovirus), of a plasmid containing a sequence encoding the ETS inhibitor bordered by two AAV inverted repeat regions (ITR), and of a plasmid carrying the AAV encapsulation genes (rep and cap genes). The recombinant AAVs produced are then purified by conventional techniques.

As regards the herpesviruses and the retroviruses, the construction of recombinant vectors has been widely described in the literature: see especially Breakfield et al., New Biologist 3 (1991) 203; EP 453242, EP 178220, Bernstein et al. Genet. Eng. 7 (1985) 235; McCormick, BioTechnology 3 (1985) 689, and the like. In particular, the retroviruses are integrative viruses which selectively infect the dividing cells. They therefore constitute vectors of interest for cancer applications. The genome of retroviruses essentially comprises two LTRs, an encapsulation sequence and three coding regions (gag, pol and env). In the recombinant vectors derived from retroviruses, the gag, pol and env genes are generally deleted, completely or partly, and replaced by a heterologous nucleic acid sequence of interest. These vectors can be prepared from various types of retroviruses such as especially MoMuLV (murine Moloney leukaemia virus, also called MoMLV), MSV (murine Moloney sarcoma virus), HaS^{t′} (Harvey sarcoma virus), SNV (spleen necrosis virus), RSV (Rous sarcoma virus) or alternatively Friend's virus.

To construct the recombinant retroviruses containing a sequence of interest, a plasmid containing especially the LTRs, the encapsulation sequence and the said sequence of interest is generally constructed and then used to transfect a so-called encapsulation cell line capable of providing in trans the retroviral functions which are deficient in the plasmid. Generally, the encapsulation lines are therefore capable of expressing the gag, pol and env genes. Such encapsulation lines have been described in the prior art, and especially the PA317 line (U.S. Pat. No. 4,861,719), the PsiCRIP line (WO 90/02806) and the GP+envAm−12 line (WO 89/07150). Moreover, the recombinant retroviruses may contain modifications in the LTRs so as to suppress the transcriptional activity, as well as extended encapsulation sequences containing a portion of the gag gene (Bender et al., J. Virol. 61 (1987) 1639). The recombinant retroviruses produced are then purified by conventional techniques. In the specific case of the present invention, the retroviruses can be prepared by homologous recombination between a retroviral plasmid containing the gene encoding GAD and a transcomplementing cell line which will make it possible to produce viral particles whose genome encodes the transgene. By way of example of a cell line, there may be mentioned especially the cell line Ψ-2 which is obtained from the NIH-3T3 line (mouse fibroblast line) by transfection with pMOV-Ψ⁻, a plasmid containing the murine Moloney leukaemia virus (MoMuLV) whose encapsulation sequence “Ψ” is deleted (Mann et al., Cell, 33, 153-159, 1983).

For the implementation of the present invention, it is most particularly advantageous to use an adenovirus or a defective recombinant retrovirus. The results given below demonstrate indeed the particularly advantageous properties of adenoviruses and retroviruses for the expression in vivo of a protein having a GAD activity. Furthermore, the adenoviral and retrovirus vectors according to the invention have, in addition, major advantages such as especially their very high efficiency of infection of nerve cells, which makes it possible to carry out infections from small volumes of viral suspension. Furthermore, infection with these viral vectors of the invention is highly localized at the site of injection, thereby avoiding the risks of diffusion to the neighbouring cells.

Advantageously, in the vectors of the invention, the sequence encoding the protein having a GAD activity is placed under the control of signals permitting its expression in nerve cells. Preferably, they are heterologous expression signals, that is to say signals different from those naturally responsible for the expression of GAD. They may be in particular sequences responsible for the expression of other proteins, or synthetic sequences. In particular, they may be promoter sequences of eucaryotic or viral genes. For example, they may be promoter sequences derived from the genome of the cell which it is desired to infect. Likewise, they may be promoter sequences derived from the genome of a virus, including the virus used. In this respect, there may be mentioned for example the ElA, MLP, CMV, RSV-LTR promoters and the like. In addition, these expression sequences can be modified by the addition of activator sequences, regulatory sequences or sequences permitting a tissue-specific expression. It may indeed be particularly advantageous to use expression signals which are active specifically or predominantly in nerve cells, such that the DNA sequence is expressed and produces its effect only when the virus has indeed infected a nerve cell. In this respect, neuron-specific enolase promoters (Forss-Petter et al., Neuron 5 (1990) 187), GFAP promoters (Sarkar et al., J. Neurochem. 57 (1991) 675) and the like can be mentioned.

In a specific embodiment, the invention relates to a defective recombinant virus comprising a cDNA sequence encoding a protein having a GAD activity under the control of the RSV-LTR promoter.

In another specific embodiment, the invention relates to a defective recombinant virus comprising a gDNA sequence encoding a protein having a GAD activity under the control of the RSV-LTR promoter.

The applicant has indeed shown that the LTR promoter of the Rous sarcoma virus (RSV) allowed a prolonged and substantial expression of the GAD activity in the cells of the human, especially central, nervous system.

Still in a preferred embodiment, the invention relates to a defective recombinant virus comprising a DNA sequence encoding a protein having a GAD activity under the control of a promoter permitting predominant expression in the nervous system.

The expression is considered as predominant for the purposes of the invention when, even if a residual expression is observed in other cell types, the expression levels are higher in nerve cells.

As indicated above, the present invention also relates to any use of a virus as described above for the preparation of a pharmaceutical composition intended for the treatment and/or prevention of neurodegenerative diseases. More particularly, it relates to any use of these viruses for the preparation of a pharmaceutical composition intended for the treatment and/or prevention of Parkinson's disease, Alzheimer's disease, amyotrophic lateral sclerosis (ALS), Huntington's disease, epilepsy, cerebrovascular degeneration and the like.

The present invention also relates to a pharmaceutical composition comprising one or more defective recombinant viruses as described above. These pharmaceutical compositions may be formulated for topical, oral, parenteral, intranasal, intravenous, intramuscular, subcutaneous, intraocular or transdermal administration and the like. Preferably, the pharmaceutical compositions of the invention contain a pharmaceutically acceptable vehicle for an injectable formulation, especially for a direct injection into the patient's nervous system. This may be in particular isotonic sterile solutions, or dry, especially freeze-dried, compositions which, upon addition, depending on the case, of sterilized water or physiological saline, permit the preparation of injectable solutions. Direct injection into the patient's nervous system is advantageous because it makes it possible to concentrate the therapeutic effect at the level of the affected tissues. Direct injection into the patient's central nervous system is advantageously carried out by means of a stereotaxic injection apparatus. The use of such an apparatus makes it possible, indeed, to target the injection site with great precision. Localized injections, such as especially intranigral injections, into the temporal lobe, into the epileptic foci or alternatively into the intestinal epithelium may be particularly advantageous.

The doses of defective recombinant virus used for the injection can be adjusted according to various parameters, and especially according to the viral vector, the mode of administration used, the pathology concerned or alternatively the desired duration of treatment. Generally, the recombinant adenoviruses according to the invention are formulated and administered in the form of doses of between 10⁴ and 10¹⁴ pfu/ml, and preferably 10⁶ to 10¹⁰ pfu/ml. The term pfu (plaque forming unit) corresponds to the infectivity of a virus solution, and is determined by infecting an appropriate cell culture, and measuring, generally after 48 hours, the number of plaques of infected cells. The techniques for determining the pfu titre of a viral solution are well documented in the literature. As regards the adenovirases, the compositions according to the invention may directly contain the producing cells, so that they can be implanted.

The GABA release obtained by gene therapy according to the invention constitutes a therapeutic approach which is particularly effective for blocking the excitotoxic phenomena responsible for degenerative nervous diseases such as amyelotrophic scleroses.

The vectors of the invention therefore have anticonvulsant and neuroprotective properties. Among the therapeutic applications of the vectors of the invention, there may be mentioned in particular the treatment of certain forms of epilepsy, especially those resistant to any pharmacological or surgical treatment, such as rebellious epilepsy of the temporal lobe. The vectors of the invention can also be used for the treatment of excitotoxic cerebral lesions of various origins, especially of ischaemic origin, as well as for the treatment of medullary traumas capable of causing excitotoxic lesions. They are also of major therapeutic value in the treatment of diabetes, especially type I, for example by inducing tolerance to GAD in prediabetic individuals, thus making it possible to avoid the autoimmune attack responsible for the symptoms of this disease. In this regard, a massive but well localized synthesis of GAD in the intestine for example is also of definite therapeutic value in cases of declared diabetes, by depressing the cellular autoimmune response.

Another subject of the invention relates to any mammalian cell infected by one or more defective recombinant viruses as described above. More particularly the invention relates to any human cell population infected by these viruses. This may be in particular fibroblasts, myoblasts, hepatocytes, keratinocytes, endothelial cells, glial cells and the like.

The cells according to the invention can be obtained from primary cultures. These can be collected by any technique known to persons skilled in the art and then cultured under conditions permitting their proliferation. As regards more particularly fibroblasts, these can be easily obtained from biopsies, for example according to the technique described by Ham [Methods Cell. Biol. 21a (1980) 255]. These cells can be used directly for infection by viruses, or preserved, for example by freezing, for establishing autologous libraries, for subsequent use. The cells according to the invention can also be secondary cultures which are obtained for example from pre-established libraries (see for example EP 228458, EP 289034, EP 400047, EP 456640).

The cultured cells are then infected with the recombinant viruses, so as to confer on them the capacity to produce a GAD activity. The infection is carried out in vitro according to techniques known to persons skilled in the art. In particular, depending on the type of cells used and the desired copy number of virus per cell, persons skilled in the art can adjust the multiplicity of infection and optionally the number of cycles of infection performed. It is clearly understood that these steps should be carried out under appropriate sterile conditions when the cells are intended for administration in vivo. The recombinant virus doses used for the infection of the cells can be adjusted by persons skilled in the art according to the desired aim. The conditions described above for administration in vivo can be applied to infection in vitro.

Another subject of the invention relates to an implant comprising mammalian cells infected with one or more defective recombinant viruses as described above, and an extracellular matrix. Preferably, the implants according to the invention comprise 10⁵ to 10¹⁰ cells. More preferably, they comprise 10⁶ to 10⁸ cells.

More particularly, in the implants of the invention, the extracellular matrix comprises a gelling compound and optionally a support permitting anchorage of the cells.

For the preparation of the implants according to the invention, various types of gelling agents can be used. The gelling agents are used for the inclusion of the cells in a matrix having the constitution of a gel, and to enhance the anchorage of the cells on the support, where appropriate. Various cell adhesion agents can therefore be used as gelling agents, such as especially collagen, gelatin, glucosaminoglycans, fibronectin, lectins, and the like. Preferably, collagen is used in the framework of the present invention. This may be collagen of human, bovine or murine origin. More preferably, type I collagen is used.

As indicated above the compositions according to the invention advantageously comprise a support permitting anchorage of the cells. The term anchorage designates any form of biological and/or chemical and/or physical interaction resulting in the adhesion and/or binding of the cells onto the support. Moreover, the cells can either cover the support used, or penetrate inside this support, or both. The use of a solid, non-toxic and/or biocompatible support is preferred within the framework of the invention. In particular, it is possible to use polytetrafluoroethylene (PTFE) fibres or a support of biological origin.,

The implants according to the invention can be implanted at different sites in the body. In particular, the implantation can be carried out in the peritoneal cavity, in the subcutaneous tissue (suprapubic region, iliac and inguinal fossae, and the like), in an organ, a muscle, a tumour, the central nervous system or alternatively under a mucous membrane. The implants according to the invention are particularly advantageous in the sense that they make it possible to control the release of the therapeutic product in the body: this release is first determined by the multiplicity of infection and by the number of implanted cells. Next, the release can be controlled either by the removal of the implant, which permanently stops the treatment, or by the use of regulable expression systems, which make it possible to induce or to repress the expression of the therapeutic genes.

The present invention thus offers a very effective means for the treatment or prevention of neurodegenerative diseases. It is most particularly adapted to the treatment of Alzheimer's disease, Parkinson's disease, Huntington's disease, epilepsy and ALS. In addition, this treatment may apply both to man and to any animal such as ovines, bovines, domestic animals (dogs, cats and the like), horses, fish and the like.

The present invention will be more completely described with the aid of the following examples and figures which should be considered as illustrative and non-limiting.

### LEGEND TO THE FIGURES

FIG. 1: Structure of the vector pLTRIXGAD67

FIGS. 2a and 2b: Infection of HiB5 and ST14a cell lines by a Ψ-2 clone producing recombinant retroviruses encoding GAD.

FIG. 3: Glutamate decarboxylase activity of various clones derived from cell lines infected with a recombinant retrovirus encoding GAD.

FIGS. 4 and 5: Control of the transplantation in vivo of the HiB5 (FIG. 4B) and St14a (FIGS. 5A and 5B) clones expressing GAD compared with uninfected ST14a cells (FIG. 4A).

FIG. 6: Release of GABA in vitro by genetically modified cells.

### GENERAL MOLECULAR BIOLOGY TECHNIQUES

The methods conventionally used in molecular biology, such as preparative extractions of plasmid DNA, centrifugation of plasmid DNA in caesium chloride gradient, agarose or acrylamide gel electrophoresis, purification of DNA fragments by electroelution, phenol or phenol-chloroform extraction of proteins, ethanol or isopropanol precipitation of DNA in saline medium, transformation in Escherichia coli and the like, are well known to persons skilled in the art and are widely described in the literature [Maniatis T. et al., “Molecular Cloning, a Laboratory Manual”, Cold Spring Harbor Laboratory, Cold Spring Harbor, N.Y., 1982; Ausubel F. M. et al. (eds), “Current Protocols in Molecular Biology”, John Wiley & Sons, New York, 1987].

The pBR322- and pUC-type plasmids and the phages of the M13 series are of commercial origin (Bethesda Research Laboratories).

For the ligations, the DNA fragments can be separated according to their size by agarose or acrylamide gel electrophoresis, extracted with phenol or with a phenol/chloroform mixture, precipitated with ethanol and then incubated in the presence of phage T4 DNA ligase (Biolabs) according to the recommendations of the supplier.

The filling of the protruding 5′ ends can be performed with the Klenow fragment of E. coli DNA polymerase I (Biolabs) according to the specifications of the supplier. The destruction of the protruding 3′ ends is performed in the presence of phage T4 DNA polymerase (Biolabs) used according to the recommendations of the manufacturer. The destruction of the protruding 5′ ends is performed by a controlled treatment with S1 nuclease.

Site-directed mutagenesis in vitro by synthetic oligodeoxynucleotides can be performed according to the method developed by Taylor et al. [Nucleic Acids Res. 13 (1985) 8749-8764] using the kit distributed by Amersham.

The enzymatic amplification of the DNA fragments by the so-called PCR technique [Polymerase-catalyzed Chain Reaction, Saiki R. K. et al., Science 230 (1985) 1350-1354; Mullis K. B. and Faloona F. A., Meth. Enzym. 155 (1987) 335-350] can be performed using a DNA thermal cycler (Perkin Elmer Cetus) according to the specifications of the manufacturer.

The verification of the nucleotide sequences can be performed by the method developed by Sanger et al. [Proc. Natl. Acad. Sci. USA, 74 (1977) 5463-5467] using the kit distributed by Amersham.

### EXAMPLES

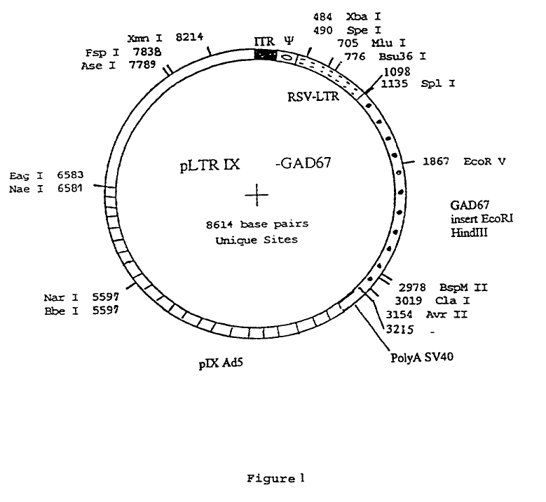

### Example 1. Construction of the vector pLTR IX GAD67 carrying the gene encoding GAD67 under the control of the Rous sarcoma virus LTR promoter (FIG. 1).

This example describes the construction of a vector comprising a cDNA sequence encoding GAD67, under the control of a promoter consisting of the Rous sarcoma virus LTR (RSV-LTR).

1.1. Starting vector (pLTR IX): the vector pLTR IX contains in particular the left region of the Ads adenovirus comprising the ITR and the encapsulation site, the RSV LTR promoter, and a region of Ad5 adenovirus stretching from the pIX gene up to the EagI restriction site, permitting homologous recombination in vivo. This vector was described by Stratford-Perricaudet et al. (J. Clin. Invest. 90 (1992) 626).

1.2. Construction of a cDNA sequence encoding GAD67.

To allow the preparation of vectors according to the invention, a cDNA sequence encoding rat GAD67 was constructed as follows:

a cDNA clone corresponding to the glutamate decarboxylase GAD67 mRNA was isolated by immunological screening of a rat brain cDNA expression library constructed in the vector lambda GT11 (Julien et al., Neurosci. Letters 73 (1987) 173). The nature and the exact position of the sequence encoding GAD67 in the isolated clone was determined by sequencing the insert. The insert contained in this clone was then isolated by digesting with the enzyme EcoRI, and then subcloned into the corresponding site of the vector pSPT18 or Bluescript (Pharmacia).

1.3. Construction of the vector pLTR IX GAD67

This example describes the construction of the vector pLTR IX GAD67 containing the sequence encoding GAD67 under the control of the RSV virus LTR, as well as Ad5 adenovirus sequences permitting recombination in vivo.

The 2 kb EcoRI-HindIIl fragment was isolated by enzymatic digestion from the construct prepared in Example 1.2. This 2 kb fragment contains the sequence encoding GAD67, and stretches to the 118 bp upstream of the codon for initiation of translation, and to the 70 bp situated downstream of the stop codon. This fragment was isolated and purified by LMP (Low Melting Point) agarose gel electrophoresis and then treated with T4 DNA polymerase to give blunt ends. This fragment was then inserted into the SalI compatible site of the vector pLTR IX (Example 1.1.) to generate the vector pLTR IX GAD67 (FIG. 1). The entire nucleotide sequence of the GAD67 insert was then checked by dideoxynucleotide sequencing.

### EXAMPLE 2. Functionality of the vector pLTR IX GAD67

The capacity of the vector pLTR IX GAD67 to express in cell culture a biologically active form of GAD was demonstrated by transient transfection of the cells 293. For this, the cells (2×10⁶ cells per dish 10 cm in diameter) were transfected (8 μg of vector) in the presence of Transfectam. The expression of the sequence encoding GAD and the production of a biologically active protein are demonstrated by the following tests:

detection and quantification of GAD mRNA by the Northern blotting technique (see for example Julien et al., Neurosci. Letters 73 (1987) 173 for a detailed procedure)

visualization of the GAD protein by the Western blotting technique using the K2 polyclonal antibody (Chemicon) (see also Julien et al., Neurosci. Letters 73 (1987) 173 for a detailed procedure)

demonstration and quantification of the GAD activity (synthesis of GABA) using the cell lysate. For this, 48 hours after the transfection, the cells are lysed and the lysate is clarified by centrifugation at 100,000 g. The lysate is then incubated in the presence of an anti-GAD antibody, itself attached to sepharose beads. The antibody used is in particular the antibody #1440, which has the property of preserving the catalytic activity of GAD (Oertel et al., Neurosci. 6 (1981) 2715). It is understood that other antibodies having comparable properties can be used and optionally prepared. The antibody-active GAD complex is then separated from the cell lysate and incubated in the presence of [1-⁴C]L-glutamate. The ¹⁴CO₂ released during the reaction, which reflects the conversion of glutamate to GABA, is trapped on a filter impregnated with hyamine and counted by liquid scintillation. Two negative controls make it possible to subtract the background noise and thus to determine the specific signal: one preimmune antiserum and one GAD-specific inhibitor, acetylenic-γ-GABA (see also Julien et al., Neurosci. Letter 73 (1987) 173).

### EXAMPLE 3: Construction of a recombinant adenovirus Ad-GAD67 containing a sequence encoding GAD67

The vector pLTR IX GAD67 is linearized and cotransfected with a deficient adenoviral vector, into the helper cells (line 293) providing in trans the functions encoded by the adenovirus El regions (ElA and ElB).

More specifically, the Ad-GAD67 adenovirus was obtained by homologous recombination in vivo between the mutant adenovirus Ad-d11324 (Thimmappaya et al., Cell 31 (1982) 543) and the vector PLTR IX GAD67, according to the following procedure: The plasmid PLTR IX GAD67 and the Ad-d11324 adenovirus, linearized by the enzyme ClaI, were cotransfected into the line 293 in the presence of calcium phosphate, so as to allow the homologous recombination. The recombinant adenoviruses thus generated were selected by plaque purification. After isolation, the recombinant adenovirus DNA was amplified in the cell line 293, thereby giving a culture supernatant containing the unpurified recombinant defective adenovirus having a titer of about 10¹⁰ pfu/ml.

The viral particles are then purified by centrifugation on a caesium chloride gradient according to known techniques (see especially Graham et al., Virology 52 (1973) 456). The Ad-GAD67 adenovirus can be preserved at −80° C. in 20% glycerol.

### EXAMPLE 4: Functionality of the Ad-GAD67 adenovirus

The capacity of the Ad-GAD67 adenovirus to infect cells in culture and to express in the culture medium a biologically active form of GAD67 was demonstrated by infecting the human lines 293. The presence of active GAD67 in the culture supernatant was then determined under the same conditions as in Example 2.

These studies make it possible to show that the adenovirus indeed expresses a biologically active form of GAD67 in cell culture.

### EXAMPLE 5: Transfer in vivo of the GAD67 gene by a recombinant adenovirus

This example describes the transfer of the GAD67 gene in vivo by means of an adenoviral vector according to the invention. It shows how the effects of the vectors of the invention on various animal models can be demonstrated.

The recombinant adenovirus GAD67 can be injected into various points of the nervous system. In particular, the injection is carried out into the following different points: into the median nucleus of the septum, into the dorsal part of the hippocampus, into the striatum, into the black substance of the entorhinal cortex.

The adenovirus injected is the Ad-GAD67 adenovirus prepared in Example 3, used in purified form (3.5×10⁶ pfu/μl) in a phosphate buffered saline solution (PBS). The injections are carried out with the aid of a cannula (external diameter 280 μm) connected to a pump. The injection rate is fixed at 0.5 μl/min, after which, the cannula remains in place for 4 additional minutes before being withdrawn.

The injection volumes into the hippocampus, the septum, the striatum and the black substance are 3, 2, 2×3 and 2 μl respectively. The adenovirus concentration injected is 3.5×10⁶ pfu/μl.

For the injection into the hippocampus, the stereotaxic coordinates are the following: AP=−4; ML=3.5; V=−3.1 (the AP and ML coordinates are determined relative to the bregma, the V coordinate relative to the surface of the cranial bone in the bregma).

For the injection into the septum, the stereotaxic coordinates are the following: AP=1; ML=1; V=−6 (the AP and ML coordinates are determined relative to the bregma, the V coordinate relative to the surface of the cranial bone in the bregma). Under this condition, the cannula is at an angle of 9 degrees relative to the vertical (in the mediolateral direction) in order to avoid the median venous sinus.

For the injection into the black substance, the stereotaxic coordinates are the following: AP=−5.8; ML=+2; V=−7.5 (the AP and ML coordinates are determined relative to the bregma, the V coordinate relative to the dura mater).

For the injections into the striatum, the stereotaxic coordinates are the following: AP=+0.5 and −0.5; ML=3; V=−5.5 (the AP and ML coordinates are determined relative to the bregma, the V coordinate relative to the dura mater).

The therapeutic effects of the administration of the adenovirus according to the invention are demonstrated by three types of analysis: a histological and immunohistochemical analysis, a quantitative analysis and a behaviloural analysis, which reflect the capacity of the vectors of the invention to produce in vivo a biologically active GAD.

### EXAMPLE 6. Construction of the retroviral plasmid pMoMuLV-GAD carrying the gene encoding GAD 67

The rat glutamate decarboxylase cDNA which was inserted into a retroviral plasmid comprises 163 non-coding base pairs in 5′ and 377 in 3′. The open reading frame encoding GAD contains 1782 base pairs. This cDNA was inserted between the two LTRs of the vector, under the transcriptional control of the 5′ LTR. The retroviral sequence also contains a sequence y which permits its encapsulation. Finally, the plasmid contains an ampicillin resistance gene which makes it possible to select the bacteria after their transformation.

Schematic representation of the recombinant retroviral sequence of the plasmid:

[chemical structure]

This rat GAD 67 cDNA was extracted from the plasmid pY21-GAD by SalI/PstI cleavage to generate a 2.32 kilobase fragment comprising the sequence encoding GAD. 28 mg of the plasmid pY21-GAD were cleaved with 5 units of each enzyme for one hour at 37° C. After digestion, DNA was precipitated with ethanol and then the GAD insert was purified after electrophoresis on a 1% low-melting point agarose gel.

The vector pMoMuLV-TH which comprises the retroviral sequences necessary for the transcription and for the integration (“LTR” and “Ψ” respectively) were cleaved by the enzymes SalI and NsiI. This cleavage made it possible to remove the tyrosine hydroxylase cDNA from the vector and to generate a cloning site for the GAD cDNA (the NsiI and PstI enzymes form compatible cohesive ends). 10 mg of DNA were cleaved with 5 enzyme units for one hour at 37° C. The vector was purified by the same method as the GAD cDNA.

90 ng of the GAD cDNA and 360 ng of the vector pMoMuLV were ligated overnight at 16° C. in the presence of 4 units of bacteriophage T4 DNA ligase.

Half of the ligated DNA was introduced into competent E. coli bacteria (XL1 blue) by electroporation and the transformant cells were selected on LB/agarose Petri dishes containing 100 mg/ml of ampicillin. The recombinant pMoMuLV-GAD clones were identified by digestion with the HindIII and KpnI enzymes which cleave the vector and the insert asymmetrically. Analysis of 24 bacterial clones by HindIII/KpnI cleavage made it possible to isolate 4 of them corresponding to the expected plasmid. One of these clones was used for the production of the retroviral plasmid.

The plasmid pMoMuLV-GAD was prepared by purification of the plasmid DNA from a recombinant clone on CsCl gradient according to the Birnboim and Doly method (Nucleic Acids Research, Vol 7: 1513-1523, 1979).

### EXAMPLE 7. Transfection of the retrovirus-producing cells.

For this step, we used the Ψ-2 cell line which has a provirus expressing all the retroviral functions required for the encapsulation of the retroviral transcripts but which is defective for the Ψ encapsulation signal. Because of this deletion, the provirus transcripts are not encapsulated. On the other hand, a retrovirus defective for the encapsulation functions but possessing a sequence y can be encapsulated by this trans-complementing cell.

The Ψ-2 cells were cotransfected with the plasmid pMoMuLV-GAD and a plasmid containing a neomycin resistance gene (pUC-SVNEO) with the aid of a calcium phosphate precipitate (Chen and Okayama, Molecular and Cellular Biology, Vol 7:2745-2752, 1988). A precipitate comprising 20 mg of pMoMuLV-GAD and 2 mg of pUC-SVNEO was left for 18 hours in contact with 10⁶ cells. Next, the culture was continued in DMEM medium (Gibco) supplemented with antibiotics (penicillin: 100 units/ml; streptomycin: 100 mg/ml) and with foetal calf serum (10% vol vol).

Two days after transfection, the cells were passed over seven Petri dishes (10 cm in diameter) and cultured in the same medium supplemented with 600 mg/ml of neomycine in order to select the transfected cells.

Fifteen days after the beginning of the selection, the neomycin-resistant clones are collected with a pasteur pipette and cultivated in 25 cm² flasks.

When the cells of the neomycine-resistant Ψ-2 clones are confluent, are placed in contact with fresh medium for 12 hours. This medium, which contains the retroviruses produced by the Ψ-2 cells, is harvested, frozen in liquid nitrogen and stored at −80° C. until used.

### EXAMPLE 8. Screening of the Ψ-2 clones by infection of cells of the NIH-3T3 line.

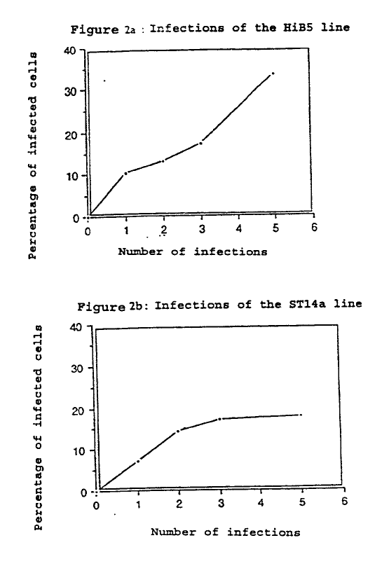

In order to determine the quality and the quantity of the retroviruses produced by the various Ψ-2 clones, the latter were tested indirectly by studying their infectious potential on cells of the NIH-3T3 line (fibroblast type cell line immortalized from mouse fibroblasts). For this, the retroviral supernatants of 35 Ψ-2 clones were used to infect the cells, and, after infection, they were either harvested in order to carry out a Western blotting or fixed in order to detect the GAD by immunocytochemistry (FIG. 2). The results of these experiments allowed us to select the Ψ-2 clone having the highest infectious potential.

### EXAMPLE 9. Infection of HiB5 and ST14a cell lines by the retroviruses

The retroviral supernatants obtained from the Ψ-2 clone, selected in Example 8, were used to infect two progenitor cell lines of the central nervous system HiB5 and ST14a:

HiB5: cell line derived from a rat hippocampus primary culture (16th day of embryonic life) immortalized by SV40 heat-sensitive T antigen. The cells of this line are progenitors for neuronal and glial type cells (Renfranz et al., Cell Vol 66: 713-729, 1991),

ST14a: this line was immortalized in the same manner as the preceding line but it is derived from a rat striatum primary culture.

The cells of the HiB5 and ST14a lines have the property of dividing in vitro at 33° C. but stop multiplying once transplanted in the central nervous system of rodents whose body temperature is 39° C. These cells can therefore be easily cultured in vitro (at 33° C.) and are not tumorigenic in vivo.

They must not be confluent during the infection: cell division (and the DNA synthesis which it causes) is necessary for the integration of the retroviral genome into the genome of the host cell (Lo et al., Molecular Neurobiology Vol 2: 156-182, 1988).

The medium is changed for the retroviral supernatant to which 10 mg/ml of polybrene has been added. The cells are left in contact with the retroviral supernatant for twelve hours and then the medium is changed for normal medium.

Given that these cells are not easily infected, use is made of a repeated infection method: the cells were infected five times at the rate of once per day for five days. The percentage of infected cells was determined by immunocytochemistry after each infection.

After five infections, the percentage of infected cells was 33% for the HiB5 line and 17% for the ST14a line (FIGS. 2a and 2b).

We undertook the subcloning of these infected cell lines in order to obtain cellular clones expressing GAD. Petri dishes (10 cm in diameter) were inoculated with the infected cells at the rate of 1000, 500 or 200 cells per dish. The isolated cellular clones were collected after 15 days of culture. We isolated 17 clones derived from the HiB5 line and 20 from the ST14a line.

### EXAMPLE 10. Functionality of the pMoMuLV-G retrovirus

The enzymatic activity of GAD is evaluated by the Hamel et al. method (Journal of Neurochemistry Vol 39: 842-849, 1982) modified by Brass et al. (Journal of Neurochemistry Vol 59: 415-424, 1992). The principle of this enzymatic activity measurement is based on the detection of the GABA formed from a tritiated precursor (³H-glutamate) by incubating at 37° C. the protein extract containing GAD. This reaction occurs in the presence of the cofactor for GAD (pyridoxal phosphate) and of an inhibitor of GABA transaminase so as to preserve the GABA formed. The tritiated GABA formed is then separated from the glutamate by means of an ion-exchange resin column and then the quantity of GABA formed is evaluated with the aid of a scintillation counter. The detailed procedure for the assay is the following:

The cells are lysed in Na phosphate buffer (pH=7.4) containing EDTA (10 mM), bovine serum albumin (BSA, 5 mg/l), and Triton X-100 (0.5% vol/vol).

The enzymatic reaction is carried out starting with 80 ml of cell lysate to which are added: (1) 10 ml of a solution containing pyridoxal phosphate (200 mM, cofactor for GAD) and aminoethyl isothiouronium bromide (10 mM, inhibitor of GABA transaminase); (2) 10 ml of a solution containing 5 mM non-labelled glutamate and 10⁶ dpm of purified tritiated glutamate immediately before use according to the Bird method (1976).

The reaction mixture is then incubated for 90 minutes at 37° C. The reaction is stopped by the addition of 0.5 ml of a cupric solution prepared immediately before use (CuCl₂ 6.92 g/l; Na₃PO₄ 59.6 g/l; sodium borate 21.68 g/l) which makes it possible to complex the glutamate. The suspension is kept on ice for 10 minutes for the complex to form and then the copper precipitate is removed by centrifugation (13,000 rpm, 5 minutes).

The supernatant is loaded onto a Dowex resin column (AG-1×8 acetate, Biorad) which retains the remaining glutamate. The column is washed twice with A 0.5 ml of water. The GABA produced is determined by counting the eluate dissolved in 10 ml of scintillation liquid (Aqueous Counting Scintillant, Amersham).

The reaction background noise is evaluated by replacing the cell lysate with cell-free lysis buffer. The concentration of the proteins of the cell lysate is determined according to the Bradford method (Analogical Biochemistry Vol 72: 248-254, 1976). The enzymatic activity is expressed as nmol of GABA formed/mg proteins/hour.

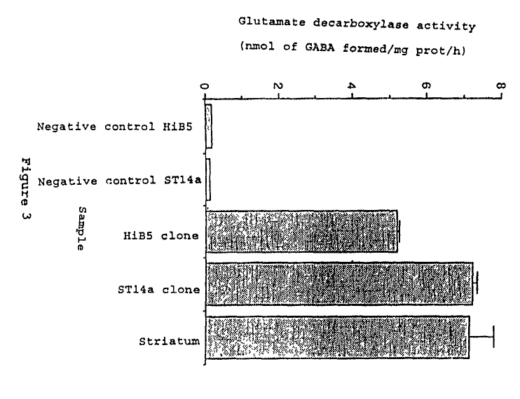

The measurement of the glutamate decarboxylase activity was carried out on each clone obtained from lines infected with the recombinant retrovirus encoding GAD. In parallel, the GAD activity was determined on extracts of striatum and of the retrovirus-producing y-2 clone. The results of the assay are presented in FIG. 3. Thus, of the 17 clones derived from the infected HiB5 cells, 3 expressed high GAD levels. Likewise, for the clones obtained from the ST14a line, 3 out of 20 expressed high GAD levels. One HiB5 clone and one ST14a clone were selected for the transplantation, their glutamate decarboxylase enzymatic activity reaching respectively:

HiB5 clone: 5.17 nmol/mg prot/hour.

ST14a clone: 7.20 nmol/mg prot/hour.

striatum: 7.13 nmol/mg prot/hour.

A Western blotting can also be carried out according to the procedure described by Towbin et al. (P.N.A.S. Vol 76: 4350-4354, 1979):

30 ml of cell lysate (same buffer as for the cellular activity) is denatured on a boiling water bath in the presence of 15 ml of “Laemmli blue” (Laemmli, Nature Vol 227: 680-685, 1970). This solution is loaded onto a 10% polyacrylamide gel containing 0.1% SDS. The gel is run overnight at 30 mV. After running, the proteins are transferred onto a nitrocellulose membrane. The GAD is revealed by immunodetection.

The immunodetections allowed us to characterize the GAD on the Western blot membranes, on the cultured cell lines, and on tissue sections after transplantation. They were carried out using GAD-immunized rabbit serum as starting material. The serum was diluted 3000 fold and incubated overnight at 4° C. The primary antibody was revealed with peroxydase (Vectastain ABC kit, Vector Laboratories). In the case of the tissue sections obtained after transplantations, the primary antibody was also revealed with a secondary antibody labelled with fluorescein (FITC goat anti-rabbit, Nordic).

### EXAMPLE 11. Transfer in vivo of the GAD67 gene by a recombinant retrovirus

The clones obtained from the HiB5 and ST14a cell lines and showing an enzymatic activity were transplanted in the striatum of Sprague-Dawley strain of rats previously anaesthetized with equitesin.

7.5×10⁵ cells taken up in 3 ml of serum-free DMEM were injected over 3 minutes by stereotaxy at the following coordinates:

anteriority: +0.6 mm relative to the bregma,

laterality: +3 mm relative to the bregma,

depth: −4.5 mm relative to the dura mater.

The needle is left in place for 3 minutes before being withdrawn.

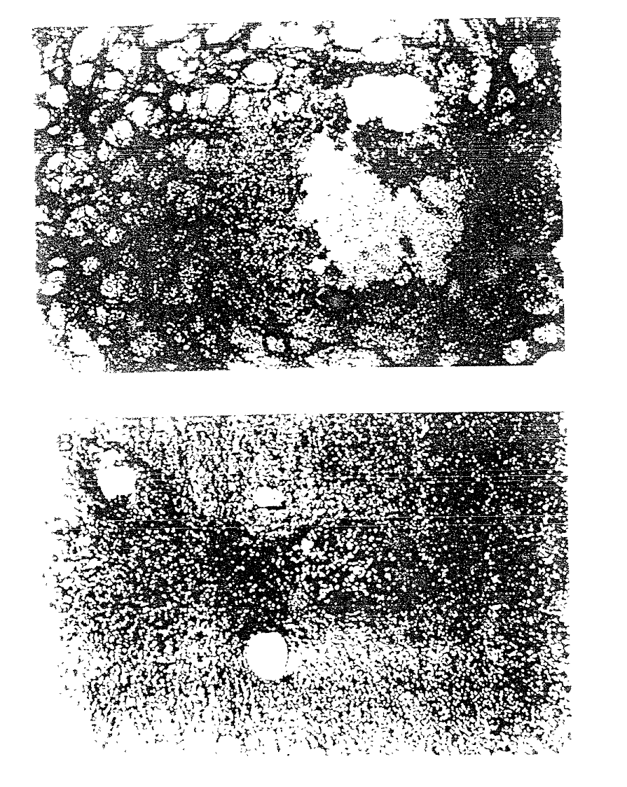

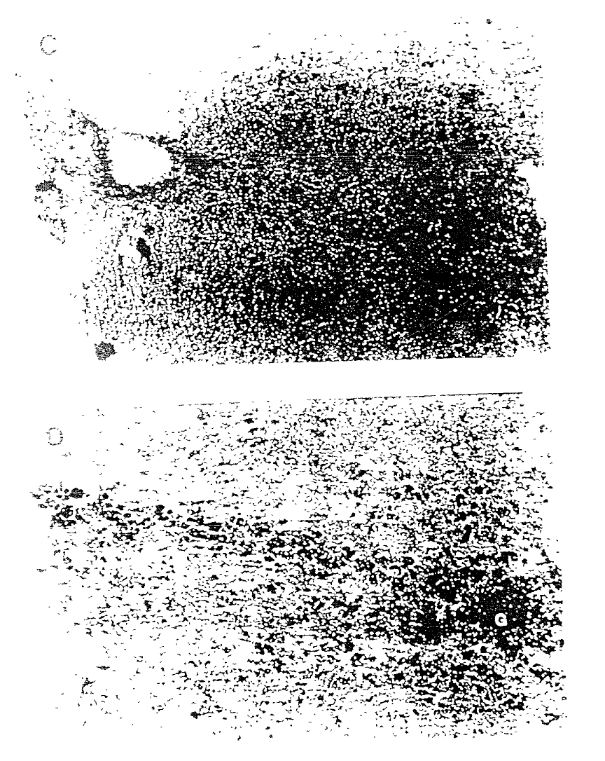

One to two weeks after transplantation, the animals were infused with a 4% paraformaldehyde solution at 4° C. and the brains were dissected and sectioned using a freezing microtome (thickness 40 mm). Immunohistochemistry was performed. The primary antibody (rabbit anti-GAD polyclonal antibody) was revealed either with a peroxydase-coupled secondary antibody, or with a fluorescent secondary antibody. We were thus able to demonstrate a GAD-specific immunolabelling in the grafts of infected HiB5 and ST14a cells compared with the grafts obtained from the uninfected cells (FIGS. 4 and 5). In FIGS. 4A are represented the uninfected ST14a cells, in 4B is presented the HiB5 clone expressing the transgene, in 5A and 5B the ST14a clone expressing the transgene. In FIGS. 4 and 5 as a whole, arrows indicate the infected cells expressing the transgene. The results demonstrate that the infected clones conserve a high level of expression of the transgene after transplantation.

### EXAMPLE 12. Release of GABA IN VITRO

The release of GABA by the HiB5-GAD and ST14a-GAD clones selected for the transplantation was studied in vitro.

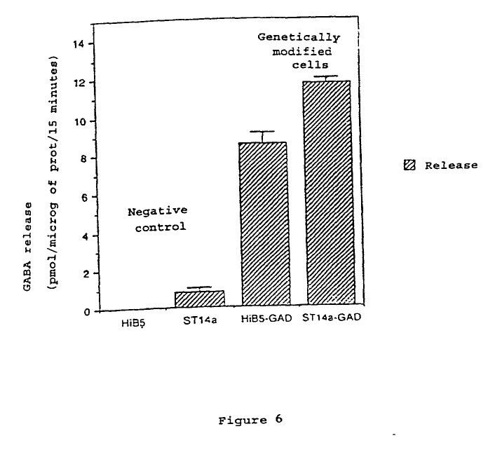

For this, the clones were cultured in a 6-well plate and then rinsed with a Hank's balanced salts type solution (HBSS, Gibco) before being incubated for fifteen minutes in the same medium. After incubation, the medium is harvested, centrifuged to remove the cells and analysed by HPLC. The cells were also harvested in order to evaluate the protein concentration. The HPLC was carried out according to the method described by Kehr and Ungerstedt (J. Neurochem. Vol 51: 1308-1310, 1988) and made it possible to demonstrate a substantial release of GABA by the cells in their culture medium. The results are presented in FIG. 6.

1. Defective recombinant virus comprising a DNA sequence encoding a protein having a glutamate decarboxylase activity (GAD).

2. Virus according to claim 1, characterized in that the DNA sequence is a cDNA sequence.

3. Virus according to claim 1, characterized in that the DNA sequence is a gDNA sequence.

4. Virus according to one of claims 1 to 3, characterized in that the DNA sequence encodes all or part of the GAD67 protein or a derivative thereof.

5. Virus according to one of claims 1 to 3, characterized in that the DNA sequence encodes all or part of the GAD65 protein or a derivative thereof.

6. Virus according to one of claims 1 to 5, characterized in that the DNA sequence is placed under the control of signals permitting its expression in nerve cells.

7. Virus according to claim 6, characterized in that the expression signals are chosen from viral promoters.

8. Virus according to claim 7, characterized in that the expression signals are chosen from the ElA, MLP, CMV and RSV-LTR promoters.

9. Defective recombinant virus comprising a cDNA sequence encoding a protein having a glutamate decarboxylase activity (GAD) under the control of the RSV-LTR promoter.

10. Defective recombinant virus comprising a gDNA sequence encoding a protein having a glutamate decarboxylase activity (GAD) under the control of the RSV-LTR promoter.

11. Defective recombinant virus comprising a DNA sequence encoding a protein having a glutamate decarboxylase activity (GAD) under the control of a promoter permitting predominant expression in the nerve cells.

12. Virus according to one of claims 1 to 11, characterized in that it lacks the regions of its genome which are necessary for its replication in the target cell.

13. Virus according to one of claims 1 to 12, characterized in that it is an adenovirus.

14. Virus according to claim 13, characterized in that it is a type Ad2 or Ad5 human adenovirus or a CAV-2 type canine adenovirus.

15. Virus according to one of claims 1 to 12, characterized in that it is an adeno-associated virus.

16. Virus according to one of claims 1 to 12, characterized in that it is a retrovirus.

17. Virus according to claim 16, characterized in that it is a retrovirus of the MoMuLV family.

18. Virus according to one of claims 1 to 12, characterized in that it is a herpesvirus (HSV).

19. Use of a virus according to one of claims 1 to 18, for the preparation of a pharmaceutical composition intended for the treatment and/or prevention of neurodegenerative diseases.

20. Use according to claim 19, for the preparation of a pharmaceutical composition intended for the treatment and/or prevention of Parkinson's disease, Alzheimer's disease, Huntington's disease, epilepsy or ALS.

21. Pharmaceutical composition comprising one or more defective recombinant viruses according to one of claims 1 to 18.

22. Pharmaceutical composition according to claim 21, characterized in that it is in injectable form.

23. Pharmaceutical composition according to claim 21 or 22, characterized in that it comprises between 10⁴ to 10¹⁴ pfu/ml, and preferably 10⁶ to 10¹⁰ pfu/ml of defective recombinant adenoviruses.

24. Mammalian cell infected with one or more defective recombinant viruses according to one of claims 1 to 18.

25. Cell according to claim 24, characterized in that it is a human cell.

26. Cell according to claim 27, characterized in that it is a human cell of the fibroblast, myoblast, hepatocyte, endothelial cell, gliala cell or keratinocyte type.

27. Implant comprising infected cells according to claims 24 to 26 and an extracellular matrix.

28. Implant according to claim 27, characterized in that the extracellular matrix comprises a gelling compound chosen preferably from collagen, gelatin, glucosaminoglycans, fibronectin and lectins.

29. Implant according to claims 27 or 28, characterized in that the extracellular matrix also comprises a support permitting anchorage of the infected cells.

30. Implant according to claim 29, characterized in that the support consists preferably of polytetrafluoroethylene fibres.
